(19) United States
(12) Patent Application Publication
(10) Pub. No.: US 2026/0077171 A1
(43) Pub. Date: Mar. 19, 2026

(54) RETRACTABLY EXPANDABLE INTRODUCER SHEATH AND ASSOCIATED METHODS

(71) Applicant: BOSTON SCIENTIFIC SCIMED, INC., Maple Grove, MN (US)

(72) Inventors: Victor Kotov, St. Louis Park, MN (US); John M. Edgell, Plymouth, MN (US)

(73) Assignee: BOSTON SCIENTIFIC SCIMED, INC., Maple Grove, MN (US)

(21) Appl. No.: 19/328,621

(22) Filed: Sep. 15, 2025

Related U.S. Application Data
(60) Provisional application No. 63/695,588, filed on Sep. 17, 2024.

(51) Int. Cl. A61M 39/02; A61B 17/34; A61M 25/00; A61M 25/06

(52) U.S. Cl. CPC A61M 39/0247; A61M 25/0045; A61M 25/0054; A61B 17/3423; A61M 2025/0004; A61M 2025/0681; A61M 2039/0258; A61M 2039/0279; A61M 2039/0291; A61M 2205/0216

(57) ABSTRACT

An introducer sheath and associated methods are disclosed herein. An example introducer sheath may include an elongate shaft having a proximal end, a distal end and a lumen extending therebetween. The elongate shaft may further include a retractably expandable section disposed between the proximal end and the distal end, the retractably expandable section may include one or more layers. At least a portion of the retractably expandable section may include a cut pattern formed in the one or more layers. The retractably expandable section may accept passage of a device through the lumen along the retractably expandable section and transition from a radially collapsed configuration before passage of the device through the retractably expandable section, to a radially expanded configuration during passage of the device through the retractably expandable section, and to a radially retracted configuration after passage of the device therethrough.

### CROSS REFERENCE TO RELATED APPLICATIONS

The present application claims the benefit of U.S. Provisional Patent Application Ser. No. 63/695,588, filed Sep. 17, 2024, the disclosure of which is incorporated herein by reference.

### TECHNICAL FIELD

The present disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient's vasculature. Inserting the medical devices into the vasculature may result in undesirable forces being applied to the vessel walls. For example, as the medical device(s) passes into the vasculature, it may make undesirable contact with one or more vessel walls. This interference may cause injury to the vessel. In some instances, an introducer sheath is utilized to facilitate the insertion of medical devices into the vessel. Further, vessel trauma resulting from forces applied to the vessel wall by medical device(s) may be lessened by minimizing the size of an introducer sheath used to access the vessel. Additionally, blood leakage may occur from the vessel and blood may escape around the outer boundaries of the introducer sheath as an operation or procedure is performed. Therefore, it may be desirable to design an introducer sheath having a reduced insertion profile while also being capable of selective expansion, contraction, and retraction to improve the efficacy and safety of various medical procedures.

Of the known medical devices and methods, each has certain advantages and disadvantages. There is an ongoing need to provide alternative medical devices as well as alternative methods for manufacturing and/or using medical devices.

### BRIEF SUMMARY

This disclosure provides design, material, manufacturing method, and use alternatives for medical device introducer sheaths and other related devices. An example introducer sheath of the present disclosure includes an elongate shaft having a proximal end, a distal end, and a lumen extending therebetween. The elongate shaft may include a retractably expandable section disposed between the proximal end and the distal end, the retractably expandable section may include an inner layer, an intermediate layer, and an outer layer. In this and other examples, at least a portion of the retractably expandable section includes a cut pattern formed in the inner layer and/or the intermediate layer. The retractably expandable section may accept passage of a device through the lumen along the retractably expandable section and transition from a radially collapsed configuration before passage of the device through the retractably expandable section, to a radially expanded configuration during passage of the device through the retractably expandable section, and to a radially retracted configuration after passage of a device through the retractably expandable section.

Alternatively or additionally to any of the examples disclosed herein, the outer layer of the retractably expandable section may include an elastomeric material elastically deformable when the retractably expandable section radially expands to the radially expanded configuration, and applies a radially inward force on the intermediate layer and the inner layer in the retracted configuration.

Alternatively or additionally to any of the examples disclosed herein, the retractably expandable section may extend along the distal end region of the elongate shaft.

Alternatively or additionally to any of the examples disclosed herein, the retractably expandable section may extend to the distal end of the elongate shaft.

Alternatively or additionally to any of the examples disclosed herein, the proximal end region of the elongate shaft may not be radially expandable.

Alternatively or additionally to any of the examples disclosed herein, the cut pattern may be a helical cut pattern.

Alternatively or additionally to any of the examples disclosed herein, the helical cut pattern may include a plurality of discontinuous slits or slots alternating with a plurality of frangible links extending in a helical direction along the retractably expandable section.

Alternatively or additionally to any of the examples disclosed herein, the plurality of frangible links may be adapted to break when the retractably expandable section radially expands from the radially collapsed configuration to the radially expanded configuration.

Alternatively or additionally to any of the examples disclosed herein, the helical cut pattern may have a varying helical pitch along a length of the retractably expandable section.

Alternatively or additionally to any of the examples disclosed herein, the cut pattern may be one or more of a helical cut pattern, a serpentine cut pattern, a sinusoidal cut pattern, a linear cut pattern, a curvilinear cut pattern, a jigsaw cut pattern or a series of perforations.

Alternatively or additionally to any of the examples disclosed herein, the retractably expandable section has an inner diameter, and the inner diameter in the radially retracted configuration may be greater than the inner diameter in the radially collapsed configuration but less than the inner diameter in the radially expanded configuration.

Alternatively or additionally to any of the examples disclosed herein, the inner diameter of the retractably expandable section in the radially collapsed configuration is 10 French or less, and the inner diameter in the radially expanded configuration is 16 French or more.

Alternatively or additionally to any of the examples disclosed herein, an introducer sheath may include an elongate shaft having a proximal end, a distal end, and an inner wall defining a lumen extending therebetween. The elongate shaft may include a retractably expandable section disposed between the proximal end and the distal end, the retractably expandable section including an inner layer, an intermediate layer, and an outer layer. At least a portion of the retractably expandable section may include a cut pattern formed in the inner layer and/or the intermediate layer. The cut pattern may include a plurality of discontinuous slits or slots alternating with a plurality of frangible links extending along the retractably expandable section. The plurality of frangible links may be adapted to break when the retractably expandable section radially expands from a radially collapsed configuration to a radially expanded configuration.

Alternatively or additionally to any of the examples disclosed herein, the outer layer of the retractably expandable section may include an elastomeric material elastically deformable when the retractably expandable section radially expands to the radially expanded configuration when subjected to a radially outward external force applied against the inner wall of the radially retractable section and the outer layer may apply a radially inward force on the intermediate layer and the inner layer to return the retractably expandable section to a retracted configuration.

Alternatively or additionally to any of the examples disclosed herein, the retractably expandable section may have an inner diameter, the inner diameter in the radially retracted configuration may be greater than the inner diameter in the radially collapsed configuration but less than the inner diameter in the radially expanded configuration.

Alternatively or additionally to any of the examples disclosed herein, methods of using medical devices are contemplated. In this and other examples, a method may include inserting a retractably expandable section of an elongate shaft of an introducer sheath into a vessel of a patient at an access site with the retractably expandable section of the elongate shaft of the introducer sheath in a radially collapsed configuration in which the retractably expandable section has an inner diameter in the radially collapsed configuration. The retractably expandable section may include an inner layer, an intermediate layer, and an outer layer. In this and other examples, a method may include advancing a medical device through a lumen of the retractably expandable section of the introducer sheath such that a distal end region of the medical device radially engages an inner wall of the retractably expandable section to expand the retractably expandable section to a radially expanded configuration to permit the distal end region of the medical device to be advanced distally beyond a distal end of the introducer sheath within the vessel. Thereafter, the retractably expandable section may be permitted to radially contract to a radially retracted configuration around a proximal region of the medical device.

Alternatively or additionally to any of the examples disclosed herein, the retractably expandable section may include a pre-formed cut pattern.

Alternatively or additionally to any of the examples disclosed herein, methods may further include advancing a dilator through the lumen of the retractably expandable section such that the dilator radially expands the retractably expandable section by widening the pre-formed cut pattern formed in the retractably expandable section.

Alternatively or additionally to any of the examples disclosed herein, the pre-formed cut pattern may include a plurality of discontinuous slits or slots alternating with a plurality of frangible links extending along the retractably expandable section. The plurality of frangible links may be adapted to break when the retractably expandable section radially expands from a radially collapsed configuration to a radially expanded configuration.

Alternatively or additionally to any of the examples disclosed herein, the pre-formed cut pattern may be a pre-formed helical cut pattern including a plurality of discontinuous slits or slots alternating with a plurality of frangible links extending in a helical direction along the retractably expandable section.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE SEVERAL VIEWS OF THE DRAWINGS

The drawings illustrate the design and utility of preferred embodiments of the present disclosure. It should be noted that the figures are not drawn to scale and that elements of similar structures or functions are represented by like reference numerals throughout the figures. In order to better appreciate how the above-recited and other advantages and objects of the present disclosure are obtained, a more particular description of the present disclosure briefly described above will be rendered by reference to specific embodiments thereof, which are illustrated in the accompanying drawings. Understanding that these drawings depict only exemplary embodiments of the disclosure and are not therefore to be considered limiting of its scope, the present disclosure will be described and explained with additional specificity and detail through the use of the accompanying drawings, in which:

FIG. 1A illustrates a perspective view of an introducer sheath with its retractably expandable section in a radially collapsed configuration.

FIG. 1B illustrates a perspective view of an introducer sheath with its retractably expandable section in a radially expanded configuration.

FIG. 1C illustrates a perspective view of an introducer sheath with its retractably expandable section in a radially retracted configuration.

FIG. 2A illustrates a side view of an introducer sheath deployed within a blood vessel with its retractably expandable section in a radially contracted configuration.

FIG. 2B illustrates a side view of an introducer sheath deployed within a blood vessel as a dilator is advanced to expand the retractably expandable section of the introducer sheath.

FIG. 2C illustrates a side view of a deployed introducer sheath after dilation, and as a medical device is being advanced through the introducer sheath.

FIG. 2D illustrates a side view of a deployed introducer sheath with its retractably expandable section in a radially retracted configuration after advancing a medical device through and out of the retractably expandable section of the introducer sheath.

FIG. 3 illustrates an enlarged partially cut-away perspective view of an introducer sheath retractably expandable section.

FIG. 4 illustrates an enlarged perspective view of an introducer sheath retractably expandable section including a cut pattern.

FIG. 5 illustrates an enlarged perspective view of an introducer sheath retractably expandable section as a dilator is guided through the retractably expandable section.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this disclosure and appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following detailed description should be read with reference to the drawings in which similar elements in different drawings are numbered the same. The drawings, which are not necessarily to scale, depict illustrative examples and are not intended to limit the scope of the disclosure.

As discussed above, inserting a medical device (e.g., a blood pump or like device) into the vasculature may result in undesirable forces being applied to the vessel walls as medical devices often have a relatively large outer diameter that is not always suitable for the inner diameter of certain vessels within a patient or subject vasculature. As the medical device passes into the vasculature, it may make undesirable contact with one or more vessel walls. This interference may cause injury to the vessel as the medical device is navigated into calcified or diseased vessels. Therefore, in some instances an introducer sheath system is utilized to position an introducer sheath into the vessel, whereby the introducer sheath is utilized to facilitate the insertion of a medical device into the vessel. For example, it may be desirable to dispose an introducer sheath along the shaft of a medical device delivery system (e.g., a percutaneous blood pump delivery system or the like), whereby the distal portion of the delivery system is withdrawn through an introducer sheath with a retractably expandable section as the system is retracted out of the body. This configuration permits the introducer sheath to remain in a state such that it applies minimal radial outward force to the inner wall of the vessel within which the introducer resides, as the retractably expandable section of the introducer sheath is capable of retracting to a diameter smaller than its expanded diameter after passage of a medical device and/or medical device system therethrough.

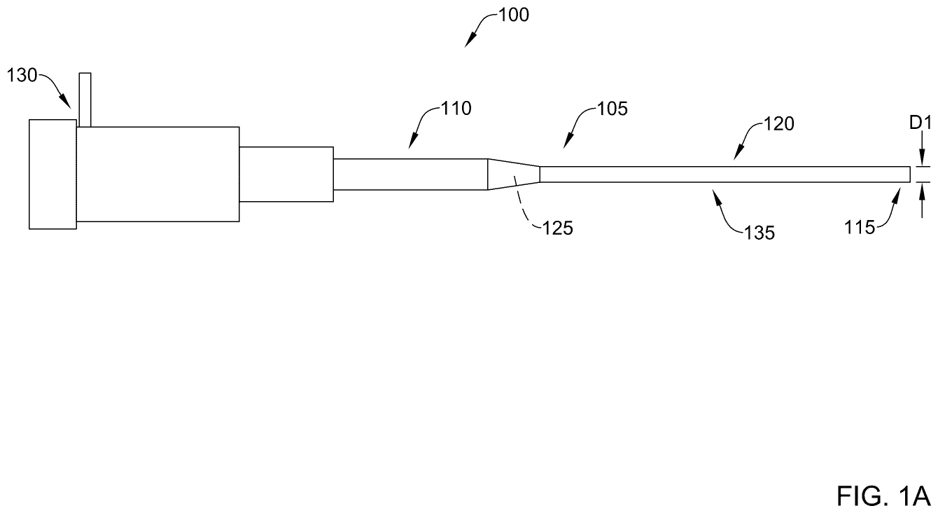

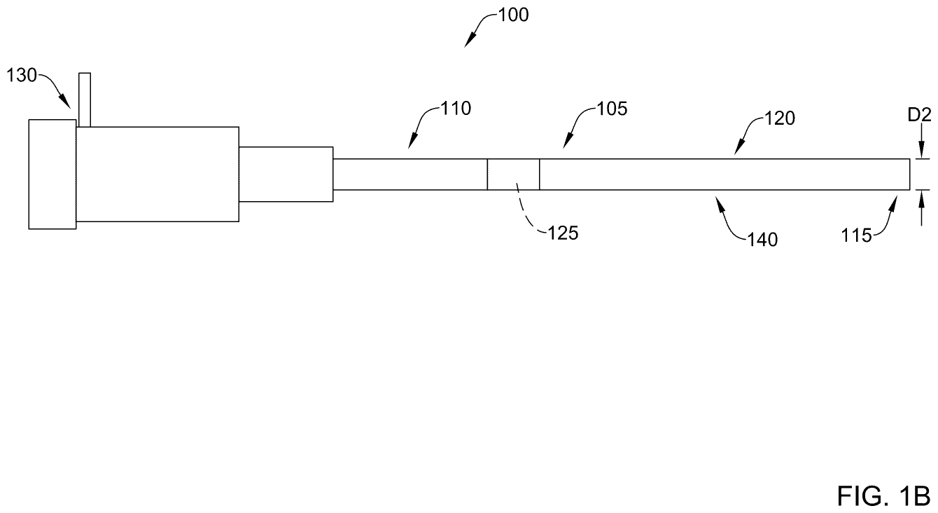

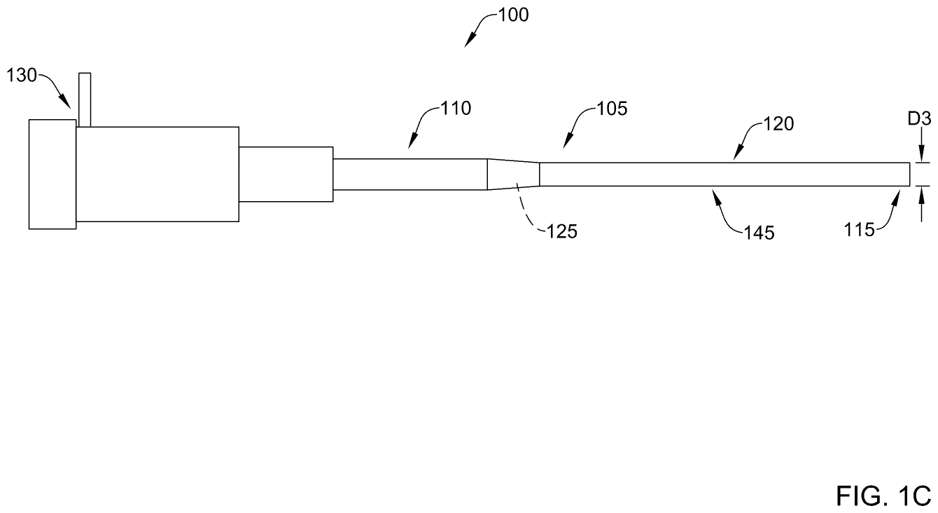

FIG. 1A is a perspective view of an example introducer sheath 100 of the present disclosure. As shown in FIG. 1A, the introducer sheath 100 may include an elongate shaft 105 having a proximal end region 110 and a distal end region 115. The proximal end region 110 may extend to and be coupled or otherwise connected to a hub 130. The distal end region 115 may extend to a distal end of the elongate shaft 105. A lumen 125 may extend through the elongate shaft 105 from the proximal end region 110 to the distal end region 115. The elongate shaft 105 may include a single lumen or a plurality of lumens disposed concentrically, coaxially, in a side-by-side relationship, in an array, or in any configuration feasible for the passage of medical devices and/or like devices. The distal end region 115 of this and any other examples may further include a distal tip defining a distal opening into the lumen 125 of the elongate shaft. In some instances, the distal tip may include radiopaque material. For instance, and applicable to all examples of the present disclosure, radiopaque material may be applied to the distal tip or any other part of the elements disclosed herein through any process known in the art.

As shown in FIG. 1A, the elongate shaft 105 of the introducer sheath 100 may further include a retractably expandable section 120, the retractably expandable section 120 may be disposed along the distal end region 115 and/or the proximal end region 110 of the elongate shaft 105. In the embodiment of FIG. 1A, the retractably expandable section 120 may extend along the distal end region 115 to the distal end of the elongate shaft 105. The lumen 125 may extend through the retractably expandable section 120. In some instances, the proximal end region 110 extending proximal of the retractably expandable section 120 may be a generally rigid, non-expandable section of the elongate shaft 105. As shown in FIG. 1A, the retractably expandable section 120 is in a radially collapsed configuration 135 having an outer diameter D₁. In the overall configuration of FIG. 1A (i.e., when the retractably expandable section 120 is in a radially contracted configuration 135), the proximal end region 110 of the elongate shaft 105 may have a relatively small outer diameter or French (Fr) size (i.e., gauge size) upon access, such as 12 Fr. However, other French sizes and gauges are contemplated for the outer diameter of the proximal end region 110 in this configuration, including but not limited to French sizes of 5 Fr of less, 6 Fr or less, 7 Fr or less, 8 Fr or less, 9 Fr or less, 10 Fr or less, 11 Fr or less, 13 Fr or less, 14Fr or less, 15 Fr or less, or other size. Further shown in the overall configuration of FIG. 1A, the retractably expandable section 120, while in the radially collapsed configuration 135, may have an outer diameter or French size and/or gauge equal to or less than that of the outer diameter or French size and/or gauge of the proximal end region 110. For instance, in some instances the retractably expandable section 120 may have a French size and/or gauge of 10 Fr. However, other French sizes and gauges are contemplated for the retractably expandable section 120 while in the radially collapsed configuration 135, including but not limited to French sizes of 3 Fr or less, 4 Fr or less, 5 Fr or less, 6 Fr or less, 7 Fr or less, 8 Fr or less, 9 Fr or less, 10 Fr or less, 11 Fr or less, 13 Fr or less, 14 Fr or less, 15 Fr or less, or other size.

FIG. 1B is a perspective view of the introducer sheath 100 of FIG. 1A with its retractably expandable section 120 in a radially expanded configuration 140 having an outer diameter D₂, wherein outer diameter D₂ is greater than outer diameter D₁. When the retractably expandable section 120 is in a radially expanded configuration 140, the distal end region 110 of the elongate shaft 105 (or at least the retractably expandable section 120 of the distal end region 110) has an expanded outer diameter or French size (i.e., gauge size) greater than its outer diameter or French size in the radially collapsed configuration 135. For example, the retractably expandable section 120 may have a French size such as 16 Fr in the radially expanded configuration. However, other French sizes and gauges are contemplated for the distal end region 115, including the retractably expandable section 120, in this configuration, including but not limited to French sizes of 10 Fr or more, 11 Fr or more, 12 Fr. or more, 13 Fr or more, 14 Fr or more, 15 Fr or more, 17 Fr or more, 18 Fr or more, 19 Fr or more, 20 Fr or more, or greater. Further shown in the overall configuration of FIG. 1B, the retractably expandable section 120, while in the radially expanded configuration 140, may have an outer diameter or French size and/or gauge equal to, less than, or greater than that of the outer diameter or French size and/or gauge of the proximal end region 110.

FIG. 1C is a perspective view of the introducer sheath 100 of FIG. 1A with its retractably expandable section 120 in a radially retracted configuration 145 having an outer diameter D₃, wherein outer diameter D₃ is less than outer diameter D₂, and in some instances, outer diameter D₃ is greater than outer diameter D₁. The radially retracted configuration 145 is a configuration in which the retractably expandable section 120 may return to upon removal of all external and/or internal forces subsequent to being radially expanded to the radially expanded configuration 140. When the retractably expandable section 120 is in a radially retracted configuration 145, the radially retracted configuration 145 may have an outer diameter or French size less than that in its radially expanded configuration, but greater than that in its radially collapsed configuration. In other words, the outer diameter of the retractably expandable section 120 in its radially retracted configuration 145 may be between the outer diameter of the retractably expandable section 120 in its radially collapsed configuration 135 and the outer diameter of the retractably expandable section 120 in its radially expanded configuration 140. In some instances, the outer diameter or French size of the retractably expandable section 120 in its radially retracted configuration 145 may be less than, equal to, or greater than the French size and/or gauge of the proximal end region 110, such as 12 Fr. However, other French sizes and gauges are contemplated for the retractably expandable section 120 while in the radially retracted configuration 140, including but not limited to French sizes of 3 Fr or less, 4 Fr or less, 5 Fr or less, 6 Fr or less, 7 Fr or less, 8 Fr or less, 9 Fr or less, 10 Fr or less, or 11 Fr or less, are contemplated. In some instances, the retractably expandable section 120 may have an outer diameter or French size in the range of 8 to 12 Fr, in the range of 9 to 12 Fr, or in the range of 10 to 12 Fr in the radially retracted configuration 145.

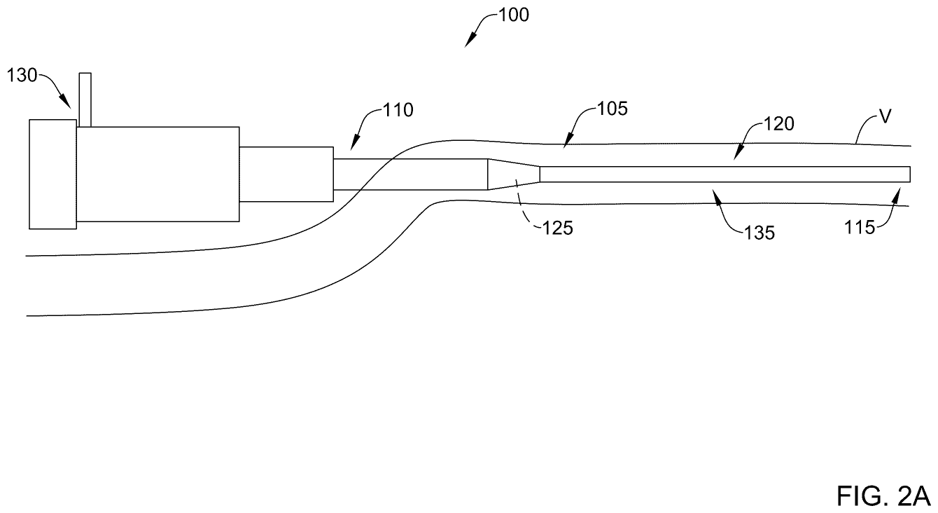

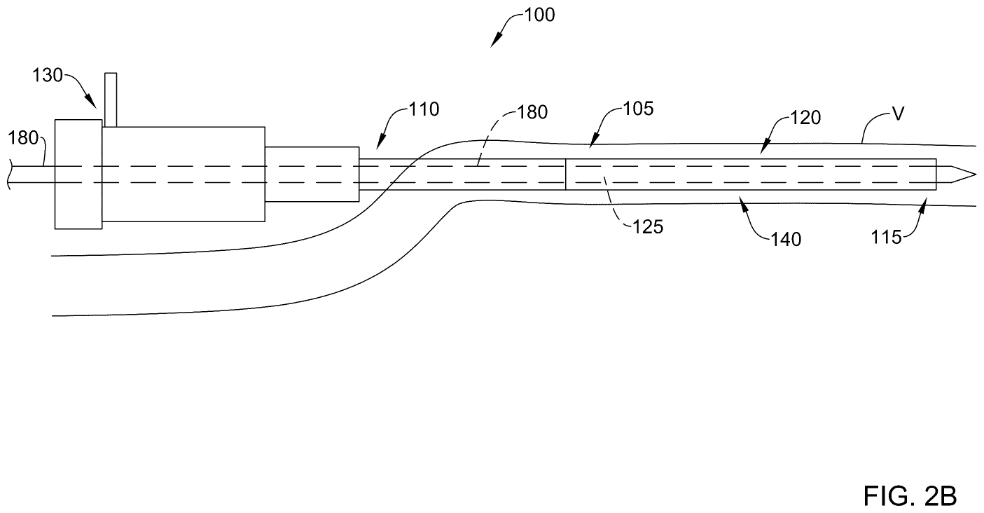

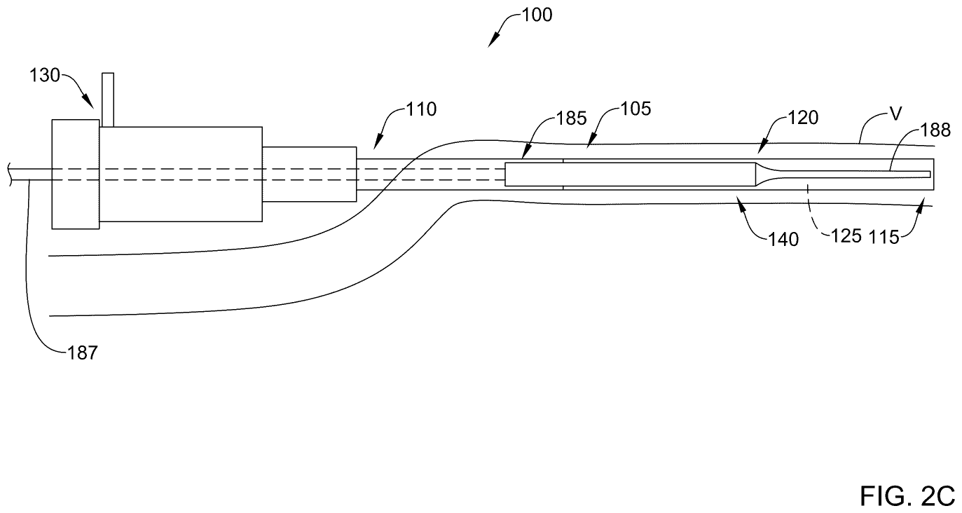

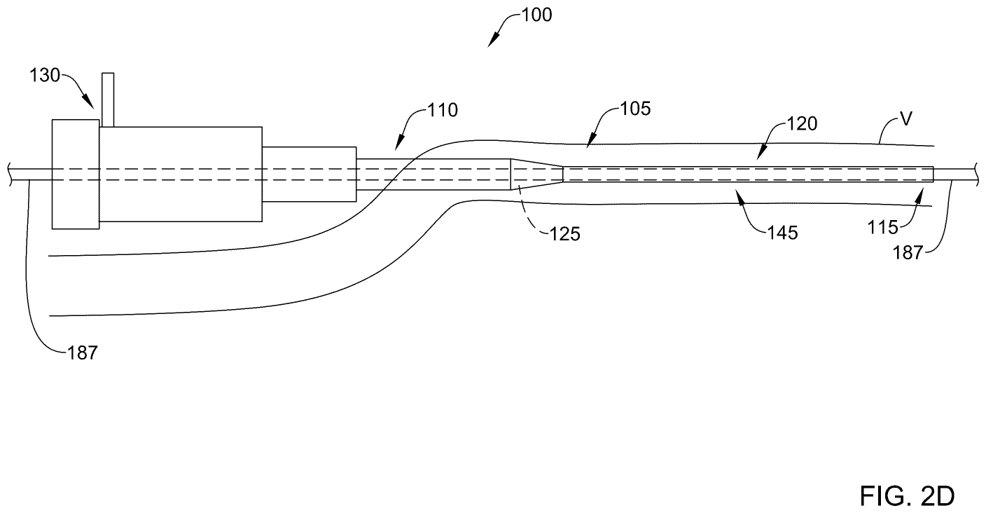

FIGS. 2A-2D schematically illustrate a sequence of steps of passing a medical device through the introducer sheath 100. FIG. 2A is a side view of the introducer sheath 100 of the present disclosure as the elongate shaft 105 of the introducer sheath 100 is positioned within the blood vessel V of a patient or subject. Following percutaneous access with a needle, cannula, guidewire, dilator or the like, an introducer sheath such as introducer sheath 100 may be deployed within a blood vessel V (e.g., by tracking along a guidewire already positioned within the blood vessel), and may be advanced into the blood vessel V with the retractably expandable section 120 of the elongate shaft 105 in a radially contracted configuration 135 within the lumen of the blood vessel V. The retractably expandable section 120 is provided in a radially contracted configuration 135 such that it provides easy access and initial navigation into the blood vessel V and subsequent vasculature prior to passage of a medical device through the introducer sheath 100.

FIG. 2B is a side view of an introducer sheath 100 of the present disclosure with a portion of the introducer sheath 100 in the next stage of deployment within the blood vessel V of a patient or subject. Further shown in FIG. 2B is dilator 180. Dilator 180 may be used to radially expand or otherwise widen the retractably expandable section 120 as will be described further herein. Dilator 180 may be any conventional dilator known in the art and may include but not be limited to a pusher tool, a stylet, a probe, or other like device and/or component.

As shown in FIG. 2B, the retractably expandable section 120 may achieve its radially expanded configuration 140 as the dilator 180 passes through the lumen 125 of the retractably expandable section 120. The dilator 180 may have an outer diameter greater than the inner diameter of the lumen 125 in the radially collapsed configuration, thereby exerting a radially outward force on the retractably expandable section 120 to radially expand the retractably expandable section 120. In other words, as the dilator 180 passes into and through the retractably expandable section 120, the portions of the dilator 180 that contact the interior wall of the retractably expandable section 120 act to expand the tubular wall of the retractably expandable section in contact with the dilator 180 to radially outwardly expand the retractably expandable section 120 into the radially expanded configuration 140. After the dilator 180 has radially expanded the retractably expandable section 120 into the radially expanded configuration 140, the dilator 180 may be removed and a medical device may be passed through thereafter, given the now larger diameter of the lumen 125 within the retractably expandable section 120 when in the radially expanded configuration 140. In other instances, a medical device may be passed through the retractably expandable section 120 without pre-expanding the retractably expandable section 120 with the dilator 180, if desired.

FIG. 2C is a side view of the introducer sheath 100 of the present disclosure with the elongate shaft 105 of the introducer sheath 100 positioned within the blood vessel V of the patient or subject after the dilator 180 or other expansion device has been withdrawn after being pushed into the retractably expandable section 120 to radially expand the retractably expandable section 120 into a radially expanded configuration 140. Also shown in FIG. 2C is a medical device, such as a percutaneous blood pump 185 being passed through the introducer sheath 100. In some instances, the blood pump 185 may be attached to a distal end of a catheter shaft 187 of the medical device to advance the blood pump 185 to a desired cardiovascular location. In some instances, the medical device may include a distal tip 188 extending distally of the blood pump 185 to be tracked over a guidewire, for example. As shown in FIG. 2C, the blood pump 185 may have an enlarged outer diameter relative to the catheter shaft 187 extending proximally of the blood pump 185 and/or the distal tip 188 extending distally of the blood pump 185. The outer diameter of the blood pump 185 may be greater than the diameter of the lumen 125 of the retractably expandable section 120 in its radially contracted configuration 135 but less than the diameter of the lumen 125 of the retractably expandable section in its radially expanded configuration 140. Given that the retractably expandable section 120 is now in its radially expanded configuration 140, a medical device, such as a blood pump 185, may be passed through the lumen 125 of the retractably expandable section 120 (after withdrawal of the dilator 180 therefrom) and further into the vasculature of the patient or subject. As the blood pump 185 or other like medical device passes through the lumen 125 of the retractably expandable section 120 in this or other configurations, the retractably expandable section 120 may remain radially expanded or radially expand further (i.e., may increase in both inner and outer diameter), particularly with regard to the portions of the retractably expandable section 120 that are in intimate contact with the medical device passing therethrough, such as blood pump 185 or other like device.

FIG. 2D is a side view of the introducer sheath 100 of the present disclosure with a portion of the elongate shaft 105 of the introducer sheath 100 positioned within the blood vessel V of a patient or subject after blood pump 185 or other like medical device has passed through (i.e., distally beyond) the retractably expandable section 120 and further into the vasculature of the patient or subject. As shown in FIG. 2D, and since the blood pump 185 or other like medical device has passed beyond the retractably expandable section 120, leaving only the smaller diameter catheter shaft 187 passing through the introducer sheath 100 and no portion of the blood pump 185 or like medical device is in direct or intimate contact with the retractably expandable section 120 to exert a radial expansion force thereon, the retractably expandable section 120 is permitted to radially contract to a radially retracted configuration 145 in which the retractably expandable section 120 now may have a smaller inner and outer diameter (i.e., French size) than the retractably expandable section 120 had in the radially expanded configuration. It is further contemplated that portions of the retractably expandable section 120 may retract instantaneously and/or automatically, particularly in the instance and particular to the portions of the retractably expandable section 120 that are no longer in intimate contact with a medical device passing therethrough, such as blood pump 185 or other like device.

It is noted that a proximal end region of the medical device (e.g., the catheter shaft 187 extending proximally from the blood pump 185) may remain disposed in the lumen 125 of the elongate shaft 105 of the introducer sheath 100, and extend therethrough. However, the outer diameter of the catheter shaft 187 is less than the inner diameter of the lumen 125 of the retractably expandable section 120 in the radially retracted configuration, and thus the catheter shaft 187 does not exert any force of the retractably expandable section 120, and thus the retractably expandable section 120 can assume its radially retracted configuration.

Due to the reduced outer diameter of the retractably expandable section 120, increased blood flow along the exterior of the elongate shaft 105 may be permitted, to ensure adequate blood flow through the vessel V. Thus, the reduced diameter of the retractably expandable section 120 of the elongate shaft 105 may not unduly obstruct blood flow through the vessel V.

Additionally, since proximal end region 110 may now possess both a greater outer and inner diameter than the retractably expandable section 120, and since the retractably expandable section resides within the blood vessel V (i.e., in-vivo), the proximal end region 110 may act as a plug and/or seal to prevent unwanted bleeding or leakage from the patient and/or subject access site (i.e., arteriotomy site) as at least a portion of the proximal end region may reside on the other side of the access site (i.e., ex-vivo).

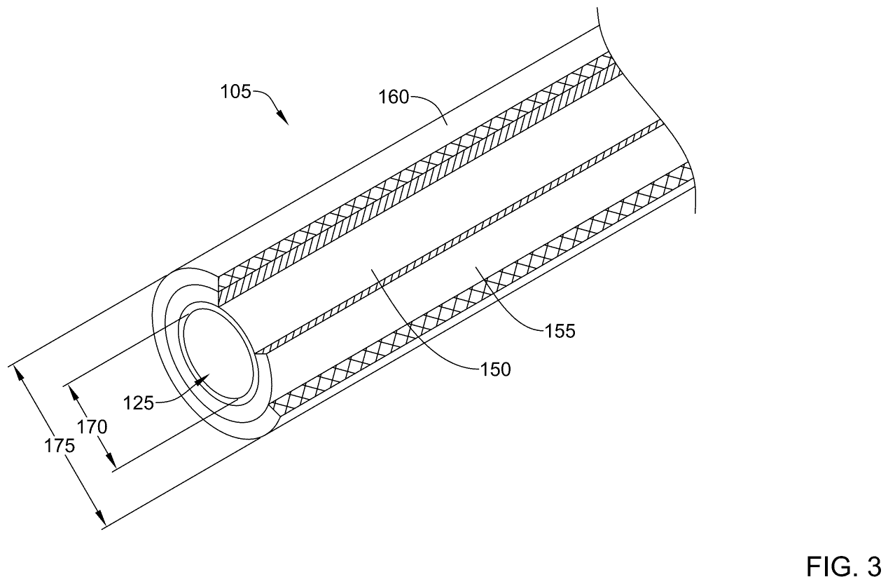

FIG. 3 is an enlarged partially cut-away perspective view of the retractably expandable section 120 of the elongate shaft 105 of the introducer sheath 100 that is applicable to any and all examples of the present disclosure. FIG. 3 shows a close-up and cutaway view of the elongate shaft 105 and its layered construction. Elongate shaft 105 overall possesses an inner diameter 170 defining the lumen 125 and an outer diameter 175. Elongate shaft 105 of this and other examples may include one or more layers, or a plurality of layers, and more particularly may include two or more layers, three or more layers, or four or more layers. In this and other examples, the elongate shaft 105 may include three layers: inner layer 150 (which may define the inner surface of the elongate shaft 105), intermediate layer 155, and outer layer 160 (which may define the outer surface of the elongate shaft 105). The intermediate layer 155 may be disposed between the inner layer 150 and the outer layer 160. As shown in FIG. 3, all layers of the elongate shaft 105 may concentrically surround the lumen 125 and longitudinal axis of the elongate shaft 105. Any of the layers described herein may be formed of the same material, different materials, a mixture of materials, a blend of materials, a patterned arrangement of materials (i.e. two layers of the same material, one layer of different material), a braid of materials, or the like. The layers may be bonded, affixed, adhered, or otherwise coupled to each other by any means or methods known in the art. Other material types, forms, and configurations are further contemplated and will be discussed herein.

The outer layer 160 of this and other examples may include an elastomeric material that is elastically deformable when the retractably expandable section 120 radially expands to the radially expanded configuration 140, and may apply a radially inward force on the intermediate layer 155 and the inner layer 150 when the retractably expandable section 120 is in the radially retracted configuration 145 by virtue of the material properties of the elastomeric material. In other words, the outer layer 160 of this and other examples may be formed of one or more elastomeric materials or like materials which are elastically deformable (i.e., able to reverse and/or revert form and/or shape under applied stress) such that the outer layer 160 may apply a radially inward force on the intermediate layer 155, particularly in the instance and particular to the portions of the retractably expandable section 120 that are no longer in intimate contact with a medical device passing therethrough, such as blood pump 185 or other like device. For example, the outer layer 160 may be formed of a thermoplastic polyurethane elastomer such as Pellethane®, in some instances.

The inner layer 150 may be a highly lubricious layer in some instances to facilitate advancing a medical device therethrough. For example, the inner layer 150 may be formed of polytetrafluoroethylene (PTFE), such as etched ePTFE, in some instances.

The intermediate layer 155 may be a polymer material, such as polyamide, a polyether block polyamide (PEBA), or the like. The radial thickness of each of the inner layer 150, the intermediate layer 155, and the outer layer 160 may be selected to provide a desired impact on the radial expansion and/or radial retraction of the retractably expandable section 120.

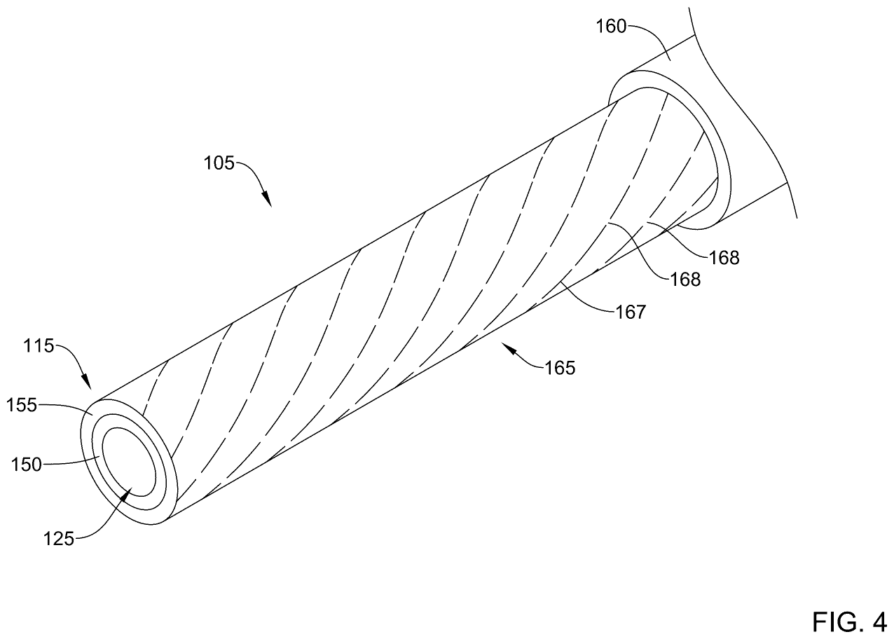

FIG. 4 is an enlarged perspective view of the introducer sheath retractably expandable section 120 with a portion of the outer layer 160 removed, to illustrate a cut pattern 165 formed in the intermediate layer 155 and/or inner layer 150 of the elongate shaft 105. As shown in FIG. 4, cut pattern 165 may extend along the retractably expandable section 120 of the elongate shaft 105 in a helical direction. However, other directions and cut patterns are contemplated. Other cut directions contemplated include, but are not limited to: serpentine cut directions, sinusoidal cut directions, hyperbolic cut directions, linear cut directions, curvilinear cut directions, lateral cut directions, radial cut directions, proximal cut directions, distal cut directions, or any combination or permutation of the aforementioned or any of the like.

The cut pattern 165 may extend uniformly along the retractably expandable section 120 in some instances. In other embodiments, the cut pattern 165 may vary along the length of the retractably expandable section 120 (e.g., the pitch of the helical slits or slots may vary along the length of the retractably expandable section 120), and/or may be discontinuous along one or more longitudinal sections of the elongate shaft 105. For example, the pitch, density, length and/or width of the helical slits or slots of the cut pattern 165 may increase in a proximal to distal direction in some instances, or the pitch, density, length and/or width of the helical slits or slots of the cut pattern 165 may decrease in a proximal to distal direction in some instances. The cut pattern 165 may be formed in the intermediate layer 155 and/or inner layer 150 prior to applying the outer layer 160. For example, the outer layer 160 may be extruded over, reflowed, or otherwise positioned over the intermediate layer 155 after forming the cut pattern 165.

Alternatively or additionally, various cut patterns are also contemplated. Cut patterns may include but are not limited to: serpentine cut patterns, sinusoidal cut patterns, hyperbolic cut patterns, jigsaw cut patterns, linear cut patterns, curvilinear cut patterns, prismatic cut patterns, geometric cut patterns, repeating cut patterns, intermittent cut patterns, or any combination or permutation of the aforementioned and/or the like.

Alternatively or additionally, any of the cut patterns described by the examples disclosed herein may be pre-formed cut patterns. In other words, any of the cut patterns described by the examples disclosed herein may be pre-set or pre-formed into one or more of the outer layer 160, intermediate layer 155, and/or inner layer 150.

The cut patterns and/or pre-formed cut patterns of the examples disclosed herein may include a plurality of discontinuous slits or slots 167 alternating with a plurality of frangible links 168 which will be further described herein. The plurality of discontinuous slits or slots 167 may alternatively or additionally take various forms including but not limited to: angled slits or slots, perforated slits or slots, curved slits or slots, helical slits or slots, curvilinear slits or slots, linear slits or slots, or any combination or permutation of the aforementioned and/or the like. Alternatively or additionally to any of the examples disclosed herein, the discontinuous slits or slots may take the non-limiting forms of perforations, depressions, grooves, etches, valleys, furrows, concavities, knurls or any combination or permutation of the aforementioned and/or the like.

As shown in FIG. 4, the cut pattern 165 may include a plurality of slits or slots 167 alternating with frangible links 168 along a helical direction. In other words, the cut pattern 165 may include a frangible link 168 between adjacent slits or slots 167 (e.g., cuts), defining discontinuous helical cuts along the cut pattern 165 of the retractably expandable section 120 of the elongate shaft 105. The frangible links 168 may be configured to break or separate as the retractably expandable section 120 is radially expanded from the radially collapsed configuration 135 to the radially expanded configuration 140 for the first time. In other instances, the links between adjacent slits or slots 167 may be made of an elastomeric material adapted to stretch and aid in the expansion of the retractably expandable section 120 into its radially expanded configuration 140 while also allowing retractably expandable section 120 to retract into its radially retracted configuration 145 by virtue of the elastomeric properties of the elastomeric or similar material incorporated as will be described further herein. It is noted that the frangible links 168 may be provided between adjacent slits or slots 167 of the cut pattern 165, regardless of the configuration of the cut pattern to permit radial expansion of the retractably expandable section 120.

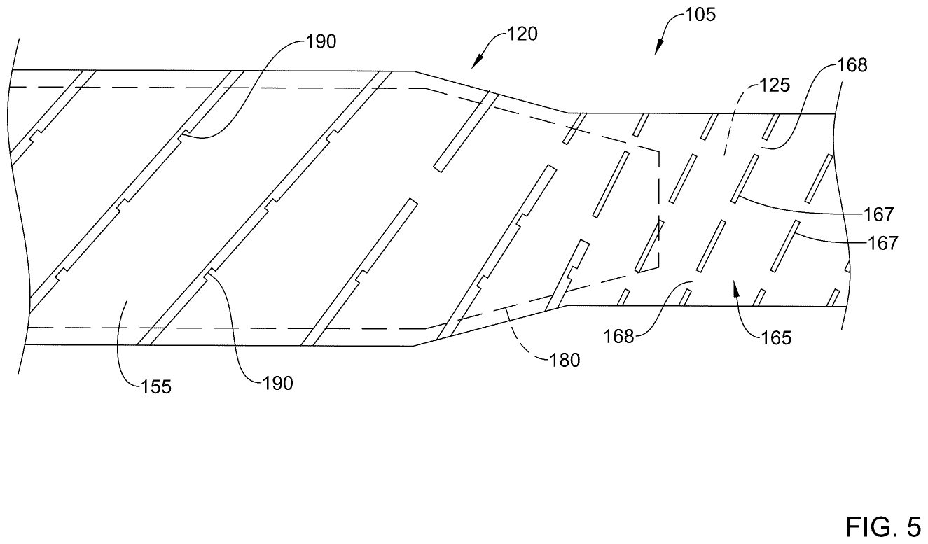

FIG. 5 is an enlarged perspective view of the retractably expandable section 120 of the introducer sheath 100 (with the outer layer 160 removed to make the cut pattern 165 visible) as a dilator 180 (i.e., pusher tool) is guided through lumen 125 of the retractably expandable section 120. The dilator 180 may be used to engage the retractably expandable section 120 from the interior of the retractably expandable section 120 and as the dilator 180 is pushed through the retractably expandable section 120, the dilator 180 contacts the interior wall of the retractably expandable section 120, expanding the slits or slots 167 of the cut pattern 165 such that they break away, and such that the frangible links 168 break apart, becoming fractured links 190. The fractured links 190, given their material construction, expand due to the internal and radial outward force exerted by the action of the dilator 180, such that the fractured links 190 may expand farther apart relative to one another and accommodate passage of a medical device such as a blood pump through the lumen of the retractably expandable section 120. Once the frangible links 168 are fractured, adjacent helical slits or slots 167 of the cut pattern 165 are united to form a continuous helical cut along the expanded portion of the retractably expandable section 120. It is noted, that, one or more, or a plurality of continuous helical cuts may be formed along the radially expanded length of the retractably expandable section 120 once the frangible links 168 have been broken or fractured. It is also noted that in some instances the helical cuts may become more parallel to the longitudinal axis along the radially expanded portion of the retractably expandable section 120 than the discontinuous helical cuts in the unexpanded portion of the retractably expandable section 120, that has not yet been radially expanded. The radial outward force exerted by the dilator 180 also radially expands and elastically stretches the outer layer 160.

As described above, after the retractably expandable section 120 is initially radially expanded to the radially expanded configuration, the radially stretched outer layer 160 may apply a radially inward force on the intermediate layer 155 and the inner layer 150 to return the retractably expandable section 120 to the radially retracted configuration 145 once the expansion force of the dilator 180 has been removed by virtue of the material properties of the elastomeric material.

Associated methods for use and utilization of all the aforementioned examples are further contemplated. An example method may include: inserting a retractably expandable section 120 of an elongate shaft 105 of an introducer sheath 100 into a vessel (V) of a patient at an access site with the retractably expandable section 120 of the elongate shaft 105 of the introducer sheath 100 in a radially collapsed configuration 135 in which the retractably expandable section 120 has an inner diameter 170 in the radially collapsed configuration 135. The retractably expandable section 120 may further include an inner layer 150, an intermediate layer 155, and an outer layer 160. Methods of this and other examples may further include advancing a medical device (such as blood pump 185) through a lumen 125 of the retractably expandable section 120 of the introducer sheath 100 such that a distal end region 115 of the medical device radially engages an inner wall of the retractably expandable section 120 to expand the retractably expandable section 120 to a radially expanded configuration 140 to permit the distal end region 115 of the medical device (such as a blood pump 185) to be advanced distally beyond a distal end 115 of the introducer sheath within the vessel (V), and thereafter, permitting the retractably expandable section 120 to radially contract to a radially retracted configuration 145 around a proximal end region 110 of the medical device.

Methods of this and other examples may further include advancing a dilator 180 (i.e., pusher tool) through the lumen 125 of the retractably expandable section 120 such that the dilator 180 radially expands the retractably expandable section 120 by widening a pre-formed cut pattern 165 formed in the retractably expandable section 120. The pre-formed cut pattern 165 of this and other examples may include a plurality of discontinuous slits or slots alternating with a plurality of frangible links 168 extending along the retractably expandable section 120. The plurality of frangible links 168 may be adapted to break when the retractably expandable section 120 radially expands from a radially collapsed configuration 135 to a radially expanded configuration 140. The pre-formed cut pattern 165 may be a pre-formed helical cut pattern including a plurality of discontinuous slits or slots alternating with a plurality of frangible links 168 extending in a helical direction along the retractably expandable section 120. In this and other examples, the pre-formed cut pattern 165 may be any pre-formed cut pattern including but not limited to: a pre-formed serpentine cut pattern, a pre-formed sinusoidal cut pattern, a pre-formed linear cut pattern, a pre-formed curvilinear cut pattern, a pre-formed hyperbolic cut pattern, a pre-formed jigsaw cut pattern, or any combination or permutation of the aforementioned or the like.

In at least some examples, portions or all of the various components of the introducer sheath 100 may also be doped with, made of, or otherwise include a radiopaque material including those listed herein or other suitable radiopaque materials. In some embodiments, a degree of MRI compatibility is imparted into the introducer sheath 100. For example, to enhance compatibility with Magnetic Resonance Imaging (MRI) machines, it may be desirable to make the various components of the introducer sheath 100 in a manner that would impart a degree of MRI compatibility. For example, the various components of the introducer sheath 100 may be made of a material that does not substantially distort the image and create substantial artifacts (artifacts are gaps in the image). Certain ferromagnetic materials, for example, may not be suitable because they may create artifacts in an MRI image. The various components of the introducer sheath 100 may also be made from a material that the MRI machine can image. Some materials that exhibit these characteristics include, for example, tungsten, cobalt-chromium-molybdenum alloys, nickel-cobalt-chromium-molybdenum alloys, nitinol, and the like, and others.

In some embodiments, the exterior surface of the introducer sheath 100 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve ease of advancement within the vasculature. Suitable lubricious polymers may include silicone and the like, polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

Some examples of suitable polymers and/or elastomers and/or elastomeric material that may be used with any of the examples disclosed herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be noted and can be appreciated that some of the FIGS. are schematic in nature and are not drawn to scale. Certain features are shown larger than their scale and certain features are omitted from some views for ease of illustration.

It should also be noted that, as used in this specification and the appended claims, the singular forms include the plural unless the context clearly dictates otherwise. It should also be noted that the term “or” is generally employed in its sense including “and/or”unless the content clearly dictates otherwise.

The present disclosure has been described with reference to various specific and exemplary embodiments. Those skilled in the art will understand that changes may be made in details, particularly in matters of shape, size, material and arrangement of parts. Accordingly, various modifications and changes may be made to the examples and the embodiments. Additional or fewer components may be used, depending on the condition that is being treated by the electrosurgical ablation device and other related devices and components disclosed herein. It should be understood that many variations and modifications may be made while remaining within the spirit and scope of the present disclosure. The specifications and drawings are, therefore, to be regarded in an illustrative rather than a restrictive sense.

What is claimed is:

1. An introducer sheath comprising:
an elongate shaft having a proximal end, a distal end, and a lumen extending therebetween;
wherein the elongate shaft includes a retractably expandable section disposed between the proximal end and the distal end, the retractably expandable section comprising an inner layer, an intermediate layer, and an outer layer;
wherein at least a portion of the retractably expandable section comprises a cut pattern formed in the inner layer and/or the intermediate layer;
wherein the retractably expandable section is configured to accept passage of a device through the lumen along the retractably expandable section and transition from a radially collapsed configuration before passage of the device through the retractably expandable section, to a radially expanded configuration during passage of the device through the retractably expandable section, and to a radially retracted configuration after passage of a device through the retractably expandable section.

2. The introducer sheath of claim 1, wherein the outer layer comprises an elastomeric material elastically deformable when the retractably expandable section radially expands to the radially expanded configuration, and applies a radially inward force on the intermediate layer and the inner layer in the retracted configuration.

3. The introducer sheath of claim 1, wherein the retractably expandable section extends along the distal end region of the elongate shaft.

4. The introducer sheath of claim 3, wherein the retractably expandable section extends to the distal end of the elongate shaft.

5. The introducer sheath of claim 4, wherein a proximal region of the elongate shaft is not radially expandable.

6. The introducer sheath of claim 1, wherein the cut pattern is a helical cut pattern.

7. The introducer sheath of claim 6, wherein the helical cut pattern includes a plurality of discontinuous slits or slots alternating with a plurality of frangible links extending in a helical direction along the retractably expandable section.

8. The introducer sheath of claim 7, wherein the plurality of frangible links are configured to break when the retractably expandable section radially expands from the radially collapsed configuration to the radially expanded configuration.

9. The introducer sheath of claim 6, wherein the helical cut pattern has a varying helical pitch along a length of the retractably expandable section.

10. The introducer sheath of claim 1, wherein the cut pattern is one or more of a helical cut pattern, a serpentine cut pattern, a sinusoidal cut pattern, a linear cut pattern, a curvilinear cut pattern, a jigsaw cut pattern or a series of perforations.

11. The introducer sheath of claim 1, wherein the retractably expandable section has an inner diameter, wherein the inner diameter in the radially retracted configuration is greater than the inner diameter in the radially collapsed configuration but less than the inner diameter in the radially expanded configuration.

12. The introducer sheath of claim 11, wherein the inner diameter in the radially collapsed configuration is 10 French or less, and the inner diameter in the radially expanded configuration is 16 French or more.

13. An introducer sheath comprising:
an elongate shaft having a proximal end, a distal end, and an inner wall defining a lumen extending therebetween;
wherein the elongate shaft includes a retractably expandable section disposed between the proximal end and the distal end, the retractably expandable section comprising an inner layer, an intermediate layer, and an outer layer;
wherein at least a portion of the retractably expandable section comprises a cut pattern formed in the inner layer and/or the intermediate layer;
wherein the cut pattern includes a plurality of discontinuous slits or slots alternating with a plurality of frangible links extending along the retractably expandable section;
wherein the plurality of frangible links are configured to break when the retractably expandable section radially expands from a radially collapsed configuration to a radially expanded configuration.

14. The introducer sheath of claim 13, wherein the outer layer comprises an elastomeric material elastically deformable when the retractably expandable section radially expands to the radially expanded configuration when subjected to a radially outward external force applied against the inner wall of the radially retractable section, and wherein the outer layer applies a radially inward force on the intermediate layer and the inner layer to return the retractably expandable section to a retracted configuration.

15. The introducer sheath of claim 14, wherein the retractably expandable section has an inner diameter, wherein the inner diameter in the radially retracted configuration is greater than the inner diameter in the radially collapsed configuration but less than the inner diameter in the radially expanded configuration.

16. A method comprising:
inserting a retractably expandable section of an elongate shaft of an introducer sheath into a vessel of a patient at an access site with the retractably expandable section of the elongate shaft of the introducer sheath in a radially collapsed configuration in which the retractably expandable section has an inner diameter in the radially collapsed configuration; wherein the retractably expandable section comprises an inner layer, an intermediate layer, and an outer layer;
advancing a medical device through a lumen of the retractably expandable section of the introducer sheath such that a distal end region of the medical device radially engages an inner wall of the retractably expandable section to expand the retractably expandable section to a radially expanded configuration to permit the distal end region of the medical device to be advanced distally beyond a distal end of the introducer sheath within the vessel;
thereafter, permitting the retractably expandable section to radially contract to a radially retracted configuration around a proximal region of the medical device.

17. The method of claim 16, wherein the retractably expandable section includes a pre-formed cut pattern.

18. The method of claim 16, further comprising:
advancing a dilator through the lumen of the retractably expandable section such that the dilator radially expands the retractably expandable section by widening the pre-formed cut pattern formed in the retractably expandable section.

19. The method of claim 16,
wherein the pre-formed cut pattern includes a plurality of discontinuous slits or slots alternating with a plurality of frangible links extending along the retractably expandable section;
wherein the plurality of frangible links are configured to break when the retractably expandable section radially expands from a radially collapsed configuration to a radially expanded configuration.

20. The method of claim 17, wherein the pre-formed cut pattern is a pre-formed helical cut pattern including a plurality of discontinuous slits or slots alternating with a plurality of frangible links extending in a helical direction along the retractably expandable section.
